(19) United States
(12) Patent Application Publication
(10) Pub. No.: US 2014/0163641 A1
(43) Pub. Date: Jun. 12, 2014

(54) MUSCLE STIMULATION SYSTEM

(71) Applicants: National University of Singapore, Singapore (SG); Agency for Science, Technology and Research, Singapore (SG)

(72) Inventors: Lei Yao, Sinapore (SG); Yong Ping Xu, Singapore (SG); Minkyu Je, Singapore (SG); Rui-Feng Xue, Singapore (SG)

(73) Assignees: National University of Singapore, Singapore (SG); Agency for Science, Technology and Research, Singapore (SG)

(21) Appl. No.: 14/099,643

(22) Filed: Dec. 6, 2013

(30) Foreign Application Priority Data
Dec. 6, 2012 (SG) 201208977-7

(51) Int. Cl. A61N 1/372; A61N 1/36

(52) U.S. Cl. CPC A61N 1/37288; A61N 1/36003
USPC 607/48

(57) ABSTRACT

According to one aspect of the invention, there is provided a muscle stimulation system for effecting appendage movement, the muscle stimulation system comprising: first transceiver circuitry; at least one first implantable stimulation device for implantation adjacent to one or more first muscles responsible for the appendage movement, the first implantable stimulation device configured to be wirelessly activated by the first transceiver circuitry; second transceiver circuitry; at least one second implantable stimulation device for implantation adjacent to one or more second muscles responsible for the appendage movement, the second implantable stimulation device configured to be wirelessly activated by the second transceiver circuitry; and a base station configured to wirelessly communicate with both the first transceiver circuitry and the second transceiver circuitry to receive and transmit signals that causes coordinated activation of the first implantable stimulation device and/or the second implantable stimulation device to coordinate stimulation of the first muscles and/or the second muscles that are responsible for the appendage movement.

### PRIORITY CLAIM

This application claims the benefit of priority of Singapore Patent Application No. 201208977-7, filed Dec. 6, 2012, the benefit of priority of which is claimed hereby, and which is incorporated by reference herein in its entirety.

### FIELD OF INVENTION

The invention relates generally to a muscle stimulation system for effecting appendage movement.

### BACKGROUND

Neuromuscular electrical stimulation system has been an active research area in biomedical field as well as in fundamental physiological study. Typically, neuromuscular electrical stimulation system comprises single or multiple stimulation devices with electrodes that generate impulses to target muscles.

Neuromuscular electrical stimulation system can serve as muscle functionality restoring tool, muscle strength testing and training tool for patients or athletes.

Conventionally, a neuromuscular electrical stimulation system uses percutaneous wires through the skin to stimulate target muscles. In order to restore muscle functionality, it is not unusual that a large number of muscles have to be stimulated in a coordinated manner. This approach involves complex surgical procedures and increases the infection risks of patient significantly. Besides that, there are also issues related to aesthetic appeal.

Wireless powered and controlled implantable devices have also been proposed to restore limited muscle functionality. This device receives power and command through a wireless inductive link which eliminates the percutaneous wire connection through the skin. The implantable device can be injected to the human muscle through a syringe. However, dexterous muscle stimulation requires small muscles to be stimulated in a localized bipolar fashion. In addition, different muscles of the human body have different stimulation strength and data transmitting modules design requirements. The implantable devices can only perform mono-polar stimulation and the stimulation capability of the implantable devices is not adjustable.

Further, due to the relatively large size, the implantable device can only be implanted in some large muscles. Therefore, it cannot be used for dexterous muscle stimulation.

A need therefore exists to provide a muscle stimulation system that seeks to address at least some of the problems above or to provide a useful alternative.

### SUMMARY

According to one aspect of the invention, there is provided a muscle stimulation system for effecting appendage movement, the muscle stimulation system comprising: first transceiver circuitry; at least one first implantable stimulation device for implantation adjacent to one or more first muscles responsible for the appendage movement, the first implantable stimulation device configured to be wirelessly activated by the first transceiver circuitry; second transceiver circuitry; at least one second implantable stimulation device for implantation adjacent to one or more second muscles responsible for the appendage movement, the second implantable stimulation device configured to be wirelessly activated by the second transceiver circuitry; and a base station configured to wirelessly communicate with both the first transceiver circuitry and the second transceiver circuitry to receive and transmit signals that causes coordinated activation of the first implantable stimulation device and/or the second implantable stimulation device to coordinate stimulation of the first muscles and/or the second muscles that are responsible for the appendage movement.

### BRIEF DESCRIPTION OF THE DRAWINGS

Example embodiments of the invention will be better understood and readily apparent to one of ordinary skill in the art from the following written description, by way of example only, and in conjunction with the drawings. The drawings are not necessarily to scale, emphasis instead generally being placed upon illustrating the principles of the invention, in which:

FIG. 1 shows a schematic representation of a muscle stimulation system in accordance to one embodiment of the invention.

FIG. 2 shows a muscle stimulation system in accordance to a preferred embodiment of the invention.

FIG. 3A shows a top perspective view of an implantable stimulation device for use with the muscle stimulation system of FIG. 2. FIGS. 3B and 3C respectively show top and bottom views of the implantable stimulation device.

### DEFINITIONS

The following provides sample, but not exhaustive, definitions for expressions used throughout various embodiments disclosed herein.

The term “appendage” may mean a body part that is joined to a larger body part such as a limb joined to its respective socket; a finger joined to its hand; a hand joined to its wrist; or a toe that is joined to its foot. Movement of such an appendage may involve the stimulation of one or more muscle groups that are required to move the appendage.

The term “transceiver circuitry” may mean a device that can both transmit and receive signals wirelessly, such as over a radio frequency.

The phrase “implantable stimulation device” may mean a device, that is implantable into a body, having electrodes that can send an electrical signal that stimulates the portion of the body where the device is implanted.

The phrase “coordinate stimulation” may mean subjecting a targeted group of muscles to an electrical signal, so that the muscles are activated and brings about desired movement of a missing body part.

### DETAILED DESCRIPTION

Disclosed is a system arrangement and device implementation of a wireless powered and coordinated functional electrical stimulation (FES) system for dexterous movement of an appendage, such as a hand. For movement of the hand, the system may comprise two separate modules; one located at a forearm and the other at the hand. Each module has two main sub-systems: 1) a wearable near filed wireless power transmitter and data transceiver (NF TRx) outside the human body; and 2) an implantable stimulation device (ISD) inside the human body. In each module, the NF TRx is powered by a battery and receives data from an external control station. The ISDs are powered and coordinated by the NF TRx through a wireless inductive link. Each module may use a different coil design and arrangement for the inductive link based on the physical constraints of human forearm and hand. The implantable stimulation device may comprise one printed circuit board (PCB) substrate, one single-channel stimulation IC with peripheral electrical components on one side of the PCB, surface stimulation electrode(s) on the other side and an inductive coil embedded in the PCB. The size and stimulation capability are scalable through the coil design and dynamic control of the ISDs.

In the following description, various embodiments are described with reference to the drawings, where like reference characters generally refer to the same parts throughout the different views.

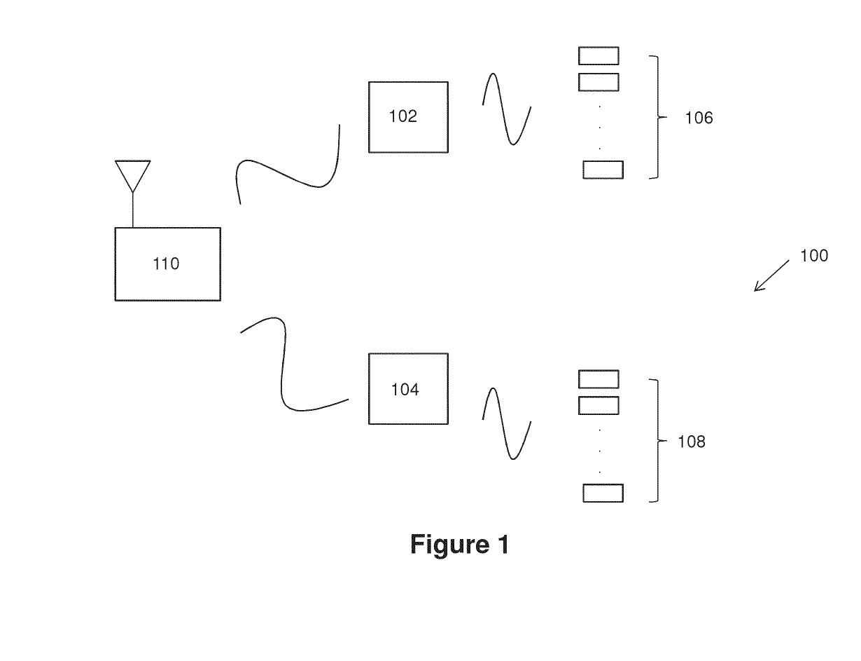

FIG. 1 shows a schematic representation of a muscle stimulation system 100 in accordance to one embodiment of the invention. The muscle stimulation system 100 is for effecting movement of an appendage (not shown). The muscle stimulation system comprises: first transceiver circuitry 102, second transceiver circuitry 104, at least one first implantable stimulation device 106, at least one second implantable stimulation device 108 and a base station 110.

The at least one first implantable stimulation device 106 is for implantation adjacent to one or more first muscles (not shown) responsible for the appendage movement. The first implantable stimulation device 106 is also configured to be wirelessly activated by the first transceiver circuitry 102.

The at least one second implantable stimulation device 108 is for implantation adjacent to one or more second muscles (not shown) responsible for the appendage movement. The second implantable stimulation device 108 is also configured to be wirelessly activated by the second transceiver circuitry 104.

The base station 110 is configured to wirelessly communicate with both the first transceiver circuitry 102 and the second transceiver circuitry 104 to receive and transmit signals that causes coordinated activation of the first implantable stimulation device 106 and/or the second implantable stimulation device 108 to coordinate stimulation of the first muscles and/or the second muscles that are responsible for the appendage movement. All control signals to the implantable stimulation devices 106 and 108 may come from the base station 110.

The number of implantable stimulation devices 106 and 108 that are activated depends on the appendage that is being moved. For example, to move a wrist and its digits to grasp, two muscles located at its forearm: the flexor digitorum profundus (FDP) and flexor pollicis longus (FPL) and muscle groups located at the hand: thenar muscles and hypothenar muscles have to be stimulated and coordinated by implanting one or more of the implantable stimulation devices 106 and 108. To perform dorsiflexion, which is a missing function of the lower limb for patients with foot drop, the muscles of the compartment (tibialis anterior, extensor hallucis longus, extensor digitorum longus and fibularis tertius) have to be stimulated by implanting one or more of the implantable stimulation devices 106 and 108. To perform synchronized eyelid movement for partial facial nerve paralysis patient, levator palpebrae superioris muscle in the orbit has to be stimulated by implanting one or more of the implantable stimulation devices 106 and 108.

In a first mode of operation, the base station 110 may only communicate with the first transceiver circuitry 102 to coordinate the activation of the required ones of the first implantable stimulation device 106, which in turn stimulates the one or more first muscles adjacent to the respective activated first implantable stimulation device 106. In a second mode of operation, the base station 110 may only communicate with the second transceiver circuitry 104 to coordinate the activation of the required ones of the second implantable stimulation device 108, which in turn stimulates the one or more second muscles adjacent to the respective activated second implantable stimulation device 108. In a third mode of operation, the base station 110 may communicate with both the first transceiver circuitry 102 and the second transceiver circuitry 104 to coordinate the activation of the required ones of the first implantable stimulation device 106 and the required ones of the second implantable stimulation device 108. This then stimulates the one or more first muscles adjacent to the respective activated first implantable stimulation device 106 and the one or more second muscles adjacent to the respective activated second implantable stimulation device 108.

To establish the coordinated activation of the first implantable stimulation device 106 and/or the second implantable stimulation device 106, each implantable stimulation device 106 and 108 may have its own address. A wireless communication link established between the base station 110 and either: the first transceiver circuitry 102, the second transceiver circuitry 104, or both, will be used to send an activation command, together with the addresses of the specific implantable stimulation devices 106 and 108 that are to be activated and coordinated to bring out the appendage movement.

The muscle stimulation system 100 may be used to control appendage movement where a neural path from the brain to the muscles that controls the appendage part is missing, due to medical conditions such as a stroke, peripheral neural damage or spinal cord injury. The input received by the muscle stimulation system 100 to control the implantable stimulation devices 106 and 108 can either be from pre-programmed algorithms or from other neural sources. For example, the muscle stimulation system 100 may include a computer (not shown) that generates appendage movement control patterns that are received by the base station 110 to transmit the signals that coordinate activation of the first implantable stimulation device 106 and/or the second implantable stimulation device 108. Alternatively, the muscle stimulation system 100 may include a neural recording device (not shown) that can capture signals from the brain/peripheral nerve terminals/spinal cord and provide them to the base station 110 to transmit the signals that coordinate activation of the first implantable stimulation device 106 and/or the second implantable stimulation device 108. Thus, the signal source of base station 110 may be from pre-programmed stimulation patterns or sorted neural recording signals.

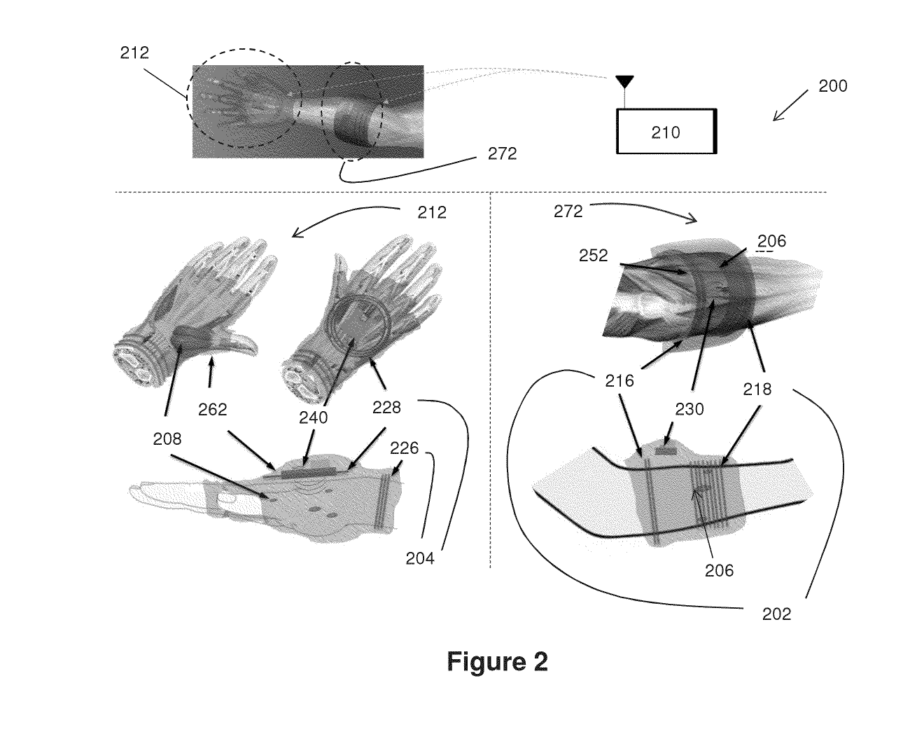

Accordingly, various embodiments of the invention are based on the schematic shown in FIG. 1, i.e. a wireless stimulation system arrangement having one implantable stimulation device (ISD), the system being for effecting appendage movement. For effecting appendage movement of, for example, a hand, at least two groups of muscle fascicles have to be stimulated. One group is located at the hand, while the other group is located at the lower arm, as shown in FIG. 2.

FIG. 2 shows a muscle stimulation system 200 in accordance to a preferred embodiment of the invention. The muscle stimulation system 200 is for effecting movement of a hand 212. The hand 212 is used for the purposes of illustration of an appendage that the preferred embodiment can be used to move. However, the muscle stimulation system 200 is suitable for effecting any other appendage movement.

Similar to the muscle stimulation system 100 of FIG. 1, the muscle stimulation system 200 comprises: first transceiver circuitry 202, second transceiver circuitry 204, at least one first implantable stimulation device 206, at least one second implantable stimulation device 208 and a base station 210.

The at least one first implantable stimulation device 206 is for implantation adjacent to one or more first muscles responsible for the hand 212 movement. These one or more first muscles may span across the lower arm, whereby an appropriate location for the first implantable stimulation device 206 may be, for example, within the forearm area that is near the elbow. The first implantable stimulation device 206 is also configured to be wirelessly activated by the first transceiver circuitry 202.

The at least one second implantable stimulation device 208 is for implantation adjacent to one or more second muscles responsible for the hand 212 movement. These one or more second muscles may span across the hand 212, whereby an appropriate location for the second implantable stimulation device 208 may be, for example, within the palm of the hand 212. The second implantable stimulation device 208 is also configured to be wirelessly activated by the second transceiver circuitry 204.

The base station 210 is configured to wirelessly communicate with both the first transceiver circuitry 202 and the second transceiver circuitry 204 to receive and transmit signals that causes coordinated activation of the first implantable stimulation device 206 and/or the second implantable stimulation device 208 to coordinate stimulation of the first muscles and/or the second muscles that are responsible for the hand 212 movement.

The first transceiver circuitry 202 may comprise an inductor 218 for data communication with the first implantable stimulation device 206 and for powering the first implantable stimulation device 206. The inductor 218 may be coupled to a radio frequency (RF) and near field (NF) transceiver processor 230, whereby the inductor 218 is configured to have near field communication with the first implantable stimulation device 206. Accordingly, the first implantable stimulation device 206 may comprise a near field receiver to receive the activation signals sent by the inductor 218 that will in turn activate the first implantable stimulation device 206 to stimulate the one or more first muscles. The first transceiver circuitry 202 may comprise an antenna 216 for the wireless communication with the base station 210 to receive command signals from the base station 210 that are meant to activate the required first implantable stimulation device 206 that brings about the desired hand 212 movement. The antenna 216 may be coupled to the processor 230 for processing the command signals from the base station 210 and determining which of the first implantable stimulation devices 206 are to be activated, for instance by matching an address embedded within the command signal to the address of the first implantable stimulation device 206. A battery compartment may be provided (not shown) that may be used to power the processor 230. The battery compartment may also be coupled to the first transceiver circuitry 202.

In the embodiment shown in FIG. 2, the inductor 218 may be realised by a helical coil, which surrounds the first implantable stimulation device 206. In another embodiment (not shown), the inductor 218 may be realised by a planar coil.

The second transceiver circuitry 204 may comprise an inductor 228 for data communication with the second implantable stimulation device 208 and for powering the second implantable stimulation device 208. The inductor 228 may be coupled to a radio frequency (RF) and near field (NF) transceiver processor 240, whereby the inductor 228 is configured to have near field communication with the second implantable stimulation device 208. Accordingly, the second implantable stimulation device 208 may comprise a near field receiver to receive the activation signals sent by the inductor 228 that will in turn activate the second implantable stimulation device 208 to stimulate the one or more second muscles. The second transceiver circuitry 204 may comprise an antenna 226 for the wireless communication with the base station 210 to receive command signals from the base station 210 that are meant to activate the required second implantable stimulation device 208 that brings about the desired hand 212 movement. The antenna 226 may be coupled to the processor 240 for processing the command signals from the base station 210 and determining which of the second implantable stimulation devices 208 are to be activated, for instance by matching an address embedded within the command signal to the address of the second implantable stimulation device 208. A battery compartment may be provided (not shown) that may be used to power the processor 240. The battery compartment may also be coupled to the second transceiver circuitry 204.

In the embodiment shown in FIG. 2, the inductor 228 may be realised by a planar coil which is in sufficient proximity for communicating with and powering the second implantable stimulation device 208. In another embodiment (not shown), the inductor 228 may be realised by a helical coil, which may surround the second implantable stimulation device 208.

The first transceiver circuitry 202 and the second transceiver circuitry 204 are respectively provided as a first wearable external module 252 and a second wearable external module 262. The external modules 252 and 262 may be provided as separate units. However, in another embodiment (not shown), the external modules 252 and 262 may be provided as an integrated unit.

The first wearable external module 252 is provided as a forearm band. In such a forearm band configuration, the inductor 218 is preferably a helical coil for fitting with the natural shape and movement of the arm when it performs near field inductive coupling with the first implantable stimulation device 206. For RF (radio frequency) communication with the base station 210, the antenna 216 may also be helix wound around the forearm band or provided as a micro strip (not shown) based on the frequency over which the RF communication occurs.

The second wearable external module 262 is provided as a glove, fitted over the hand 212. In such a glove configuration, the inductor 228 is preferably a planar spiral coil provided on the back of the hand 212, instead of the helical coil arrangement used by the inductor 218, in order not to block the free movement of the wrist and fingers. The inductor 228 also performs near field inductive coupling with the second implantable stimulation devices 208. For RF communication with the base station 210, the antenna 216 may also be helix wound around the glove or provided as a micro strip (not shown) based on the frequency over which the RF communication occurs.

From the above, the muscle stimulation system 200 shown in FIG. 2 has a base station 210 (which is preferably in a portable size), two wearable external modules (the forearm band 252 and the glove 262) on an appendage (namely an arm from the elbow to the tip of the hand) and several implantable ISDs 206 and 208. The base station 210 communicates and coordinates the two wearable modules 252 and 262 to achieve movement control of the hand 212. The forearm module 252 communicates with the base station 210, transmits command signals to the ISDs 206 in the forearm 272 and powers the ISDs 206 in the forearm 272. The hand module 262 communicates with the base station 210, transmits command signals to the ISDs 208 in the hand 212 and powers the ISDs 208 in the hand 212. The ISDs 206 and 208 perform stimulation of the targeted muscles. For each wearable external module (252, 262), an RF data transceiver (the antennas 216 and 226) and near-field power and data transceiver (the inductors 218 and 228) are embedded.

The data transmission between the base station 210 and the wearable external modules 252 and 262 uses radio frequency (RF) technology. The power transmitted from the wearable external modules 252 and 262 and the data communication between the wearable external modules 252 and 262 and the respective ISDs 206 and 208 are through a near field wireless power transmitter and data transceiver (i.e. the inductors 218 and 228) embedded in the wearable external modules 252 and 262.

To perform dexterous hand 212 functions, a multitude of different muscles scattered within the hand 212 and forearm 272 areas have to be stimulated in a coordinated manner. Thus the ISDs 206 and 208 have to be wirelessly powered to avoid battery change and minimise the risk of infection in a large number of implant sites. The implantable stimulation devices 206 and 208 are preferably also flexible in size and stimulation capability to deal with different muscles.

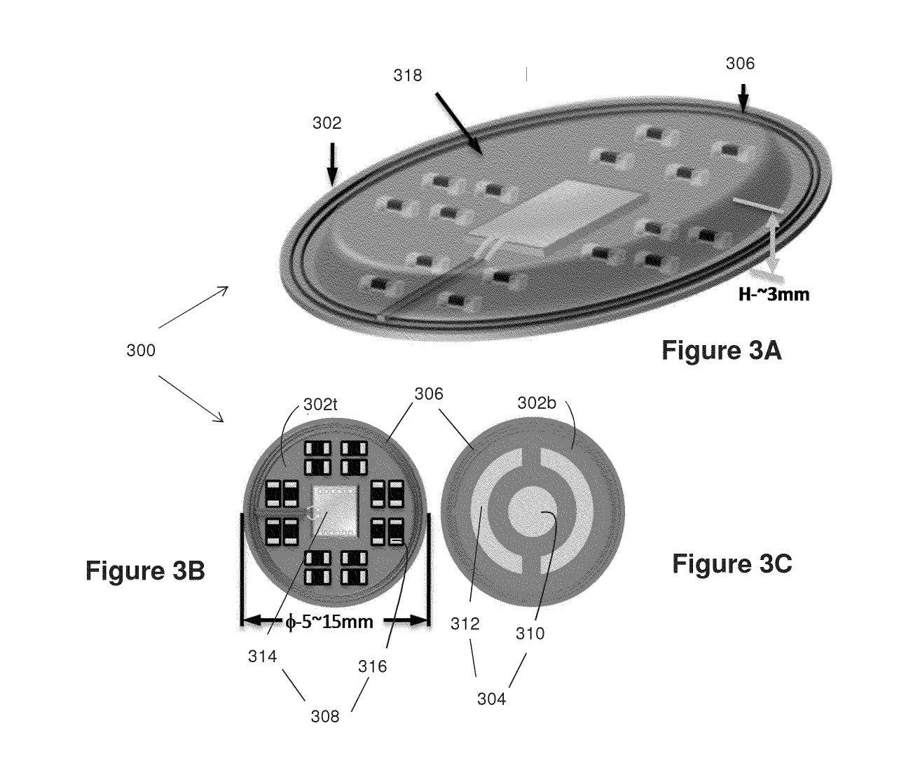

FIGS. 3A to 3C show an implantable stimulation device that meets the above criteria. FIG. 3A shows a top perspective view of an implantable stimulation device 300 for use with a muscle stimulation system in accordance to one embodiment of the invention. For example, the implantable stimulation device 300 can be used for the first implantable stimulation device 206 or the second implantable stimulation devices 208. FIGS. 3B and 3C respectively show top and bottom views of the implantable stimulation device 300. The structure of the implantable stimulation device 300 is described below with reference to FIGS. 3A, 3B and 3C.

The implantable stimulation device 300 comprises a substrate 302. An electrode arrangement 304 is provided on one side 302b of the substrate 302. The electrode arrangement 304 is for stimulation of muscles adjacent to where the implantable stimulation device 300 is located. In the case of the first implantable stimulation device 206 (see FIG. 2), the implantable stimulation device 300 will stimulate one or more first muscles (such as muscles located within the forearm 272). In the case of the second implantable stimulation device 208 (see FIG. 2), the implantable stimulation device 300 will stimulate one or more second muscles (such as muscles located within the hand 212).

An inductor 306 is coupled to the substrate 302. The inductor 306 is for electrical communication with the inductor of a transceiver circuitry. For instance, when the implantable stimulation device 300 is paired with the first transceiver circuitry 202, the inductor 306 of the implantable stimulation device 300 will be in electrical communication with the inductor 218 of the first transceiver circuitry 202. When the implantable stimulation device 300 is paired with the second transceiver circuitry 204, the inductor 306 of the implantable stimulation device 300 will be in electrical communication with the inductor 228 of the second transceiver circuitry 204.

Electronics 308 are coupled to the electrode arrangement 304. The electronics 308 are provided on the other side 302t of the substrate 302. The electronics are powered by the inductor 306 coupled to the substrate 302.

The embodiment shown in FIGS. 3A to 3C has the inductor 306 of the implantable stimulation device 300 disposed along an edge of the substrate 300 of the implantable stimulation device 300. However, in another embodiment, the inductor 306 may be located on any other part of the substrate 300.

The electrode arrangement 304 may be realised by bipolar electrodes. The bipolar electrodes may be implemented through a central electrode 310 being partially surrounded by two arc-shaped electrodes 312. It is also not essential that three electrodes 310 and 312 are used. The total number of electrodes and their arrangement may be determined by the muscles that are being activated and may therefore be less or more than three and not restricted to the bipolar arrangement shown in FIG. 3C. However, it is preferable for the electrode arrangement 304 to be the only components at the bottom side 302b to ensure close contact with muscles adjacent to where the implantable stimulation device 300 is implanted.

The electronics 308 may comprise a stimulator integrated circuit (IC) chip 314 and peripheral components 316. The IC chip 314 may be configured to regulate the wireless power received by the inductor 306 from an external inductor (such as the inductors 218 and 228 described above with reference to FIG. 2) and process the wireless data from a base station (such as the base station 210 described above with reference to FIG. 2) that cause activation of the implantable stimulation device 300. The peripheral components 316 may be external passive components required in the design of the IC chip 314, such as capacitors and inductors to provide charge storage and voltage boosting.

The size and capability of the implantable stimulation device 300 may be adjusted through design of the inductor 306 and dynamic control of the IC chip 314. The substrate 302 of the implantable stimulation device 300 may be fabricated, for example, from a biocompatible printed circuit board (PCB) made of polyimide, to serve as a main body of the implantable stimulation device 300 and provide mechanical support. The IC chip 314 may be mounted at the center on the surface of the substrate 302. The inductor 306 may, for example, be a near field coupling coil fabricated, using planar spiral arrangement, in the middle of layers of the PCB of the substrate 302, and surrounding both the IC chip 314 and the peripheral components 316. The top side 302t of the substrate 302 may have encapsulation 312, fabricated from, for example, biocompatible material, for chronic implantation and electrical insulation.

From the above, a coordinated wireless and lead-free muscle stimulation system for dexterous appendage movement control is disclosed. The disclosed muscle stimulation system has two advantages. Firstly, wireless control eliminates percutaneous wires that are required to establish coordination among different stimulation sites to enable dexterous appendage movement. Secondly, each implantable stimulation device has a stimulation IC chip and stimulation electrode(s) integrated on a same PCB board. This allows size scalability and stimulation capability for the dexterous appendage movement control required in different muscles. Integration on the same PCB board avoids the need for lead wires between the stimulation electronics and the electrodes to avoid extra tunneling in the human body, so that lead-free system is achieved.

It will be appreciated by a person skilled in the art that numerous variations and/or modifications may be made to the present invention as shown in the embodiments without departing from a spirit or scope of the invention as broadly described. The embodiments are, therefore, to be considered in all respects to be illustrative and not restrictive.

What is claimed is:

1. A muscle stimulation system for effecting appendage movement, the muscle stimulation system comprising:
first transceiver circuitry;
at least one first implantable stimulation device for implantation adjacent to one or more first muscles responsible for the appendage movement, the first implantable stimulation device configured to be wirelessly activated by the first transceiver circuitry;
second transceiver circuitry;
at least one second implantable stimulation device for implantation adjacent to one or more second muscles responsible for the appendage movement, the second implantable stimulation device configured to be wirelessly activated by the second transceiver circuitry; and
a base station configured to wirelessly communicate with both the first transceiver circuitry and the second transceiver circuitry to receive and transmit signals that causes coordinated activation of the first implantable stimulation device and/or the second implantable stimulation device to coordinate stimulation of the first muscles and/or the second muscles that are responsible for the appendage movement.

2. The muscle stimulation system according to claim 1, wherein the first transceiver circuitry comprises an inductor for data communication with the first implantable stimulation device and for powering the first implantable stimulation device.

3. The muscle stimulation system according to claim 2, wherein the inductor is a helical coil or a planar coil.

4. The muscle stimulation system according to claim 1, wherein the second transceiver circuitry further comprises an inductor for data communication with the second implantable stimulation device and for powering the second implantable stimulation device.

5. The muscle stimulation system according to claim 4, wherein the inductor is a helical coil or a planar coil.

6. The muscle stimulation system according to claim 2, wherein the first implantable stimulation device comprises:
a substrate;
an electrode arrangement provided on one side of the substrate, the electrode arrangement for stimulation of the first muscles;
an inductor coupled to the substrate, the inductor for electrical communication with the inductor of the first transceiver circuitry; and
electronics coupled to the electrode arrangement, the electronics provided on the other side of the substrate, the electronics being powered by the inductor coupled to the substrate.

7. The muscle stimulation system according to claim 6, wherein the inductor of the first implantable stimulation device is disposed along an edge of the substrate of the first implantable stimulation device.

8. The muscle stimulation system according to claim 6, wherein the electrode arrangement of the first implantable stimulation device is realized by bipolar electrodes.

9. The muscle stimulation system according to claim 4, wherein the second implantable stimulation device comprises:
a substrate;
an electrode arrangement provided on one side of the substrate, the electrode arrangement for stimulation of the second muscles;
an inductor coupled to the substrate, the inductor for electrical communication with the inductor of the second transceiver circuitry; and
electronics coupled to the electrode arrangement, the electronics provided on the other side of the substrate, the electronics being powered by the inductor coupled to the substrate.

10. The muscle stimulation system according to claim 9, wherein the inductor of the second implantable stimulation device is disposed along an edge of the substrate of the second implantable stimulation device.

11. The muscle stimulation system according to claim 9, wherein the electrode arrangement of the second implantable stimulation device is realized by bipolar electrodes.

12. The muscle stimulation system according to claim 1, wherein the first transceiver circuitry and the second transceiver circuitry are respectively provided as a first wearable external module and a second wearable external module.

13. The muscle stimulation system according to claim 12, wherein the external modules are provided as an integrated unit.

14. The muscle stimulation system according to claim 13, wherein the external modules are provided as separate units.

15. The muscle stimulation system according to claim 14, wherein the first wearable external module is provided as a forearm band.

16. The muscle stimulation system according to claim 14, wherein the second wearable external module is provided as a glove.

17. The muscle stimulation system according to claim 1, wherein the first transceiver circuitry and the second transceiver circuitry are each coupled to a respective battery compartment.

18. The muscle stimulation system according to claim 1, wherein the first transceiver circuitry and the second transceiver circuitry each comprise an antenna for the wireless communication with the base station.

19. The muscle stimulation system according to claim 1, wherein the appendage movement is selected from a group comprising: an orthotic, a prosthesis and atrophied muscles.
